(19) United States
(12) Patent Application Publication
(10) Pub. No.: US 2010/0035296 A1
(43) Pub. Date: Feb. 11, 2010

(54) COMPOUNDS AND METHODS FOR DETECTION OF CELLS

(76) Inventors: Anat Shirvan, Herzliya (IL); Ilan Ziv, Kfar Saba (IL)

Correspondence Address: Pearl Cohen Zedek Latzer, LLP, 1500 Broadway, 12th Floor, New York, NY, 10036, US

(21) Appl. No.: 12/522,108

(22) PCT Filed: Jan. 3, 2008
(86) PCT No.: PCT/IL08/00014

§ 371 Date: Jul. 2, 2009

Related U.S. Application Data
(60) Provisional application No. 60/878,407, filed on Jan. 4, 2007.

(51) Int. Cl. C12Q 1/02; C12N 5/02; C07C 69/34

(52) U.S. Cl. 435/29; 435/375; 560/190

(57) ABSTRACT

The invention relates to compounds comprising an ester group for the detection in vivo of cells undergoing cell death (“dying cells”) such as, for example, cells undergoing apoptosis. These compounds are selectively retained in dying cells relative to normal cells. Thus, the compounds may be used in the detection, diagnosis and treatment of clinical conditions manifested by a cell death process.

### FIELD OF THE INVENTION

The invention relates to compounds comprising an ester group, that are retained within biological cells undergoing death processes, such as cells undergoing apoptosis. The invention further provides methods for utilizing the compounds in medical practice, for diagnostic and therapeutic purposes.

### BACKGROUND OF THE INVENTION

Apoptosis is an intrinsic program of cell self-destruction or “suicide”, which is inherent in every eukaryotic cell. In response to a triggering stimulus, cells undergo a highly characteristic cascade of events of cell shrinkage, blebbing of cell membranes, chromatin condensation and fragmentation, culminating in cell conversion to clusters of membrane-bound particles (apoptotic bodies), which are thereafter engulfed by macrophages. It is now known that apoptosis has a role in many medical disorders, either in the etiology or pathogenesis of the disease.

In clinical practice, it is highly desirable to have chemical compounds capable of selective retention within or by cells undergoing a process of cell death. For example, if attached to a marker for imaging, such compounds may serve as useful tools for molecular imaging, allowing early detection of disease or assessment of efficacy of treatment for various medical disorders.

### SUMMARY OF THE INVENTION

In an aspect of the invention, there are provided esteric compounds, represented by the structures set forth in formulae I-VI, that are selectively retained in or by cells undergoing death process, while showing faster washout and less retention in viable cells. In some embodiments of the present invention, the cells are neuronal cells undergoing a neurodegenerative process or cells undergoing apoptosis. The invention further relates to methods of detecting the dying cells by using these compounds.

The term “dying cell” refers herein to a cell that undergoes a death process. Such cells may be exemplified by cells undergoing an apoptotic process, or neuronal cells undergoing neurodegeneration.

The term “normal cell” refers hereinbelow to cells that have a normal plasma membrane potential.

The term “dying-cell-retained-compound” (DCRC) refers to a compound that performs selective retention in dying cells, which is different from normal cells. According to the invention, binding or retention of the DCRC to the dying cell should be a least 30% higher than its binding to the normal cells. It is noted that the measurement of the DCRC should take in consideration the washout time of DCRC from normal, viable cells. The measurement can be after the washout process starts or any other time thereafter. In an embodiment of the invention, the washout time is within 10 minutes. In another embodiment, the washout time is within one hour. In another embodiment, the washout time is five hours or less. In another embodiment, the washout time is 12 hours or less. In another embodiment, the washout time from normal cells or cells that are not undergoing an apoptotic process is 24 hours. The determination of the optimal time for measuring the binding or retention of the DCRC can be calculated by one skilled in the art and is defined hereinbelow as “a certain period of time”.

The term “selective retention” refers in the invention to the selective retention of a compound in dying cells, i.e., retention in a dying-cell to an extent being at least 30% greater than the retention in normal cells, taking in consideration that the amount is determined following the washout process of the DCRC from the normal cell.

The term “diagnostic DCRC” refers to a compound capable of selective retention in dying cells, wherein the compound comprises or is linked to a marker for imaging, whereas the marker is detectable by means known to those skilled in the art.

The term “therapeutic DCRC” refers to a DCRC as defined above, comprising a drug, useful in the treatment of a disease.

The term “DCRC-PET precursor” refers to a DCRC of formula I-VI, as defined herein, comprising or being linked to a moiety. The moiety part of the precursor is to be substituted by an ¹⁸F radio-isotope upon radio-labeling, thereby generating an ¹⁸F-labeled DCRC compound, which is detectable by positron emission tomography (PET) imaging.

A DCRC may be used, according to an embodiment of the invention, for the preparation of an agent for selective retention in dying cells.

In one aspect, the invention provides a compound, which is selectively retained within or by a dying cell (i.e., a DCRC) wherein the compound is represented by the structure set forth in formula (I):

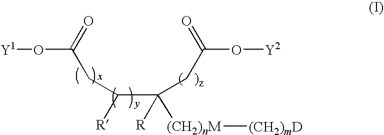

or pharmaceutically acceptable salts, metal chelates, solvates and hydrates of the compound represented by the structure set forth in formula (I), and solvates and hydrates of the salts; wherein Y¹ is selected from hydrogen, C₁, C₂, C₃, C₄, C₅ and C₆ linear or branched alkyl, or an optionally substituted aryl or heteroaryl; Y² is selected from hydrogen, C₁, C₂, C₃, C₄, C₅ and C₆ linear or branched alkyl, or an optionally substituted aryl or heteroaryl; at least one of Y¹ or Y² is other than hydrogen; each of R and R′ groups is independently selected from hydrogen, C₁, C₂, C₃, C₄, C₅, C₆, C₇, C₈, C₉, C₁₀, C₁₁, C₁₂, C₁₃, C₁₄, C₁₅, C₁₆, linear or branched alkyl, linear or branched hydroxy-alkyl, linear or branched fluoro-alkyl, aryl or heteroaryl composed of one or two rings, or combinations thereof; n and m each stands for an integer of 0, 1, 2, 3 or 4; n and m may be the same or different; M is selected from null, hydrogen, —O—, or —S—; x, and z each stands independently and is an integer of 0, 1 or 2, where x and z can be the same or different; y is an integer of 0, 1 or 2, wherein when y=2, the substituent R′ may be the same or different at each occurrence; and D is a marker for diagnostics, hydrogen, hydroxyl, or a drug; wherein the marker for diagnostics is selected from a marker for imaging such as ¹⁸F or a radio-labeled metal chelate; the marker for imaging may be detected by color, fluorescence, x-ray, CT scan, magnetic resonance imaging (MRI) or radio-isotope scan, such as single photon emission tomography (SPECT) or positron emission tomography (PET). Alternatively, D is a drug to be targeted to the dying cell. The drug may be a medicinally-useful agent for the prevention, amelioration, or treatment of a specific disease and may be, for example, without being limited: an inhibitor of apoptosis (e.g., a caspase inhibitor, antioxidant, modulator of the Bcl-2 system), or an activator of cell death (e.g. an anticancer drug). In an embodiment of the invention, there is provided a method for improvement of anti-cancer therapy, by targeting anti-cancer drugs to tumors, via targeting the drug employing the compounds of the invention to foci of apoptosis, which occur within tumors either spontaneously, or in response to therapy.

In another embodiment of the invention, there is provided a compound which is selectively retained within or by a dying cell, represented by the structure set forth in formula (II):

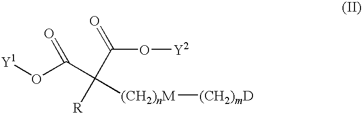

including pharmaceutically acceptable salts hydrates, solvates and metal chelates of the compound represented by the structure set forth in formula (II) and solvates and hydrates of the salts; wherein Y¹ is selected from hydrogen, C₁, C₂, C₃, C₄, C₅ and C₆ linear or branched alkyl, or an optionally substituted aryl or heteroaryl; Y² is selected from hydrogen, C₁, C₂, C₃, C₄, CS and C₆ linear or branched alkyl, or an optionally substituted aryl or heteroaryl; at least one of Y¹ or Y² is other than hydrogen; R represents hydrogen or C₁, C₂, C₃, C₄, C₅, C₆, C₇, C₈, C₉, C₁₀, C₁₁, C₁₂, C₁₃, C₁₄, C₁₅, C₁₆, linear or branched alkyl, linear or branched hydroxy-alkyl, linear or branched fluoro-alkyl, aryl or heteroaryl composed of one or two rings, or combinations thereof; n and m each stands for an integer of 0, 1, 2, 3 or 4; n and m may be the same or different; M is selected from null, hydrogen, —O—, —S—, and —N(U), wherein U stands for null, hydrogen, C₁, C₂, C₃, or C₄ alkyl; D is hydrogen or a marker for diagnostics. The marker for diagnostics may be in an embodiment of the invention a marker for imaging such as F, wherein the F may be ¹⁸F or ¹⁹F or a labeled metal chelate; the marker for imaging may be detected by color, fluorescence, x-ray, CT scan, magnetic resonance imaging (MRI) or radio-isotope scan such as single photon emission tomography (SPECT) or positron emission tomography (PET). Alternatively, D is a drug to be targeted to the dying cell, as define above.

In another embodiment of the invention there is provided a compound represented by the structure set forth in formula (III):

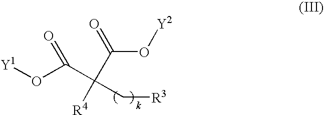

including pharmaceutically acceptable salts, hydrates, solvates and metal chelates of the compound represented by the structure set forth in formula (III) and solvates and hydrates of the salts; wherein R³ is ¹⁸F, ¹⁹F or hydroxyl; R⁴ is hydrogen, C₁, C₂, C₃, C₄, C₅, C₆, C₇, C₈, C₉ or C₁₀ linear or branched alkyl, and k is an integer selected from 0, 1, 2, 3, 4, 5, 6, 7, 8, 9 and 10; Y¹ is selected from hydrogen, C₁, C₂, C₃, C₄, C₅ and C₆ linear or branched alkyl; Y² is selected from hydrogen, C₁, C₂, C₃, C₄, C₅ and C₆ linear or branched alkyl; at least one of Y¹ or Y² is other than hydrogen. In a preferred embodiment, Y¹ and Y² are each an ethyl group.

In another embodiment of the invention there is provided a compound represented by the structure set forth in formula (IV):

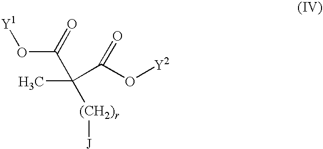

including pharmaceutically acceptable salts hydrates, solvates and metal chelates of the compound represented by the structure set forth in formula (IV) and solvates and hydrates of the salts; wherein J is —¹⁸F, —¹⁹F, —OH or a fluorescent group, and r stands for an integer of 1, 2, 3, 4, 5, 6, 7, 8, 9 and 10; Y¹ is selected from hydrogen, C₁, C₂, C₃, C₄, C₅ and C₆ linear or branched alkyl, or an optionally substituted aryl or heteroaryl; Y² is selected from hydrogen, C₁, C₂, C₃, C₄, C₅ and C₆ linear or branched alkyl, or an optionally substituted aryl or heteroaryl; at least one of Y¹ or Y² is other than hydrogen. In a preferred embodiment, Y¹ and Y² are each an ethyl group. In another preferred embodiment, J is a dansyl group.

In the case that J is a dansyl group, r=5, and Y¹ and Y² are each a methyl group, the compound is designated NST-ML-M.

In yet another embodiment of the invention, there is provided a compound represented by the structure set forth in formula (V):

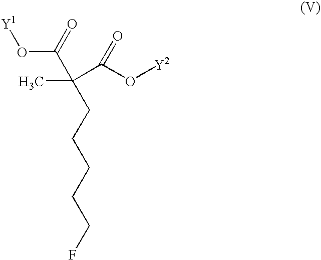

including pharmaceutically acceptable salts hydrates, solvates and metal chelates of the compound represented by the structure set forth in formula (V) and solvates and hydrates of the salts; wherein Y¹ is selected from hydrogen, C₁, C₂, C₃, C₄, C₅ and C₆ linear or branched alkyl, or an optionally substituted aryl or heteroaryl; Y² is selected from hydrogen, C₁, C₂, C₃, C₄, C₅ and C₆ linear or branched alkyl, or an optionally substituted aryl or heteroaryl; at least one of Y¹ or Y² is other than hydrogen; F is either ¹⁸F or ¹⁹F.

In yet another embodiment of the invention, there is provided a compound represented by the structure set forth in formula (VI):

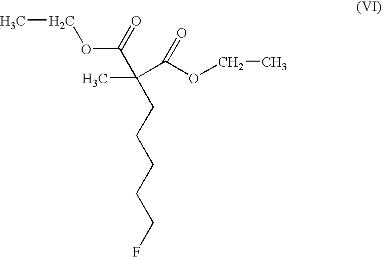

including pharmaceutically acceptable salts hydrates, solvates and metal chelates of the compound represented by the structure set forth in formula (VI) and solvates and hydrates of the salts; wherein F is either ¹⁸F, ¹⁹F.

In another aspect of the invention, there is provided a pharmaceutical composition for targeting of drugs to dying cells in a patient, wherein the patient may be a human or non-human mammal, wherein the pharmaceutical composition comprising a compound according to the structure set forth in formulae I, II, III, IV, V, or VI, wherein the compound comprises or is linked to a drug.

In an aspect of the invention, there is provided a method of selectively targeting a medicinally-useful compound to dying cells within a population of cells, the method comprising: contacting the cell population with a compound represented by the structure set forth in any one of formulae I, II, III, IV, V or VI, thereby selectively targeting the medicinally-useful compound to the dying cells within the cell population.

In another aspect of the invention, there is provided a method of detecting dying cells within a cell population, the method comprising: (i). contacting the cell population with compound represented by the structure set forth in any one of formulae I, II, III, IV, V or VI, comprising a marker for diagnostics, or pharmaceutically acceptable salts, metal chelates, solvates and hydrates of the compound represented by the structure set forth in any one formulae I, II, III, IV, V or VI and solvates and hydrates of the salts; and (ii). determining, after a certain period of time, the amount of the compound bound to the cells, wherein a significant amount of the compound bound to a cell indicates that the cell is being a dying cell.

In another aspect of the invention, there is provided a method for detecting of dying cells in a patient or an animal, the method comprising: (i). administering to the patient or animal a compound represented by the structure set forth in formulae I, II, III, IV, V or VI, comprising a marker for imaging, or pharmaceutically acceptable salts, metal chelates, solvates and hydrates of the compound represented by the structure set forth in formulae I, II, III, IV, V or VI and solvates and hydrates of the salts; and (ii) imaging the examined patient or animal, so as to determine after a certain period of time, the amount of the compound bound to cells, wherein a significant amount of compound bound to a cell indicates that the cell is a dying cell.

In another aspect of the invention, there is provided a pharmaceutical composition for targeting of drugs to foci of dying cells, for example foci of a tumor, in a patient or an animal, the pharmaceutical composition comprising a compound according to the structure set forth in formulae I, II, III, IV, V or VI, wherein the compound comprises or is linked to a drug.

### BRIEF DESCRIPTION OF THE DRAWINGS

The invention will now be described in connection with certain preferred embodiments with reference to the following illustrative figures so that it may be more fully understood.

With specific reference now to the Figures in detail, it is stressed that the particulars shown are by way of example and for purposes of illustrative discussion of the preferred embodiments of the present invention only and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1 (A, B and C) demonstrates fluorescent microscopy pictures showing selective accumulation of NST-ML-M in tumor cells (FIGS. 1A and 1B, intravenous (I.V.) administration of 70 mg/Kg and 140 mg/Kg of NST-ML-M, respectively) and in cells of the small intestine epithelium (FIG. 1C, 70 mg/Kg of I.V. NST-ML-M) undergoing apoptosis induced by the chemotherapeutic agents cyclophosphamide and Taxol.

### DETAILED EMBODIMENTS OF THE INVENTION

In the detailed description, numerous specific details are set forth in order to provide a thorough understanding of the invention. However, it will be understood by those skilled in the art that these are specific embodiments and that the invention may be practiced also in different ways that embody the characterizing features of the invention as described and claimed herein.

The invention is related to compounds, capable of performing selective retention within dying cells (DCRC), wherein the compounds are washed out faster from normal cells than from dying cells. The dying cells are cells undergoing a death process. Such cells are, in one embodiment of the invention, cells undergoing apoptosis, or cells undergoing neurodegeneration. The invention further relates to methods of detecting and imaging dying cells by using compounds, comprising or being linked to a marker for imaging.

PCT Patent Publication No. WO/2005/067388, which is incorporated herein by reference, discloses a novel class of compounds, capable of selective binding to cells undergoing a death process, e.g., by apoptosis. The present invention concerns certain ester-prodrugs of the compounds disclosed in PCT Patent Publication No. WO/2005/067388, and their use in detection of dying cells. Unlike the compounds disclosed in PCT Patent Publication No. WO/2005/067388, which are excluded from viable and/or healthy cells while performing selective entry and retention within dying cells, the ester prodrugs disclosed in the invention, are hydrophobic compounds that freely diffuse into all cells, healthy or sick, including passage through the intact blood-brain-barrier (BBB). Their performance is based on differential washout from the cells, i.e., is relative retention in dying cells while showing markedly faster washout and less retention in healthy/viable cells. This differential washout is either of the ester prodrug itself or optionally of the respective mono- or di-acid. These mono- or di-free acid forms may potentially be produced intracellularly by cleavage of the esteric bond by cellular esterases.

A potential advantage, among others, of the ester compounds of the present invention is their being uncharged as compared to the compounds disclosed in PCT Patent Publication No. WO/2005/067388. Since it is well known that uncharged moieties have better penetration through intact blood-brain-barrier (BBB) as compared to their charged analogues, the ester prodrugs of the present invention are expected to manifest facilitated penetration through the intact BBB. This may have a beneficial effect on the performance of these compounds as detectors of dying cells within the central nervous system (CNS), in medical disorders wherein BBB integrity is maintained. Another advantage of the ester compounds of the invention is in the attachment of a radio tracer, such as the ¹⁸F radio-isotope. Due to the short half-life of the ¹⁸F radio-isotope and the conditions of its attachment to the molecule for imaging, it is desirable to have as short and simple route of manufacture as possible. Since the step of attachment of the ¹⁸F requires protection of the carboxyl groups by esterification, the subsequent synthesis of the free acid forms as described PCT Patent Publication No. WO 2005/067388, requires an additional synthetic step, comprising the removal of the ester groups and conversion to the free acid form.

This deprotection step is not required for the synthesis of the ester compounds of the present invention, thus rendering the chemistry of radiolabeling for the ester compounds markedly more simple and rapid.

The compounds of the invention have the advantage of featuring a relatively low molecular weight, and a potentially favorable pharmacokinetic profile. An important advantage of the compounds of the invention is their ability to cross the intact blood-brain-barrier (BBB), thus being potentially useful in imaging dying cells within the central nervous system (CNS).

In one aspect, the invention provides a compound that is selectively retained within a dying cell (i.e., a DCRC) wherein the compound is represented by the structure set forth in formula (I):

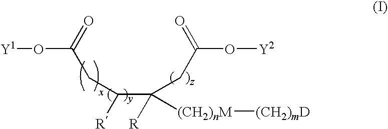

or pharmaceutically acceptable salts, metal chelates, solvates and hydrates of the compound represented by the structure set forth in formula (I), and solvates and hydrates of the salts; wherein Y¹ is selected from hydrogen, C₁, C₂, C₃, C₄, C₅ and C₆ linear or branched alkyl, or an optionally substituted aryl or heteroaryl; Y² is selected from hydrogen, C₁, C₂, C₃, C₄, C₅ and C₆ linear or branched alkyl, or an optionally substituted aryl or heteroaryl; at least one of Y¹ or Y² is other than hydrogen; each of R and R′ groups is independently selected at each occurrence from hydrogen, C₁, C₂, C₃, C₄, C₅, C₆, C₇, C₈, C₉, C₁₀, C₁₁, C₁₂, C₁₃, C₁₄, C₁₅, C₁₆, linear or branched alkyl, linear or branched hydroxy-alkyl, linear or branched fluoro-alkyl, aryl or heteroaryl composed of one or two rings, or combinations thereof; n and m each stands for an integer of 0, 1, 2, 3 or 4; n and m may be the same or different; M is selected from null, hydrogen, —O—, or —S—; x, and z each stands independently and is an integer of 0, 1 or 2, where x and z can be the same or different; y is an integer of 0, 1 or 2, where when y=2 the substituent R′ may be the same or different at each occurrence; and D is a marker for diagnostics, hydrogen, hydroxyl, or a drug; wherein the marker for diagnostics is selected from a marker for imaging, such as ¹⁸F or a radio-labeled metal chelate; the marker for imaging may be detected by color, fluorescence, x-ray, CT scan, magnetic resonance imaging (MRI) or radio-isotope scan, such as single photon emission tomography (SPECT) or positron emission tomography (PET). Alternatively, D is a drug to be targeted to the dying cells.

The drug may be a medicinally-useful agent for the prevention, amelioration, or treatment of a specific disease and may be, for example, without being limited: an inhibitor of apoptosis (e.g., a caspase inhibitor, antioxidant, modulator of the Bcl-2 system), or an activator of cell death (e.g. an anticancer drug). In an embodiment of the invention, there is provided a method for improvement of anti-cancer therapy, by targeting anti-cancer drugs to tumors, via targeting the drug, using the compounds of the present invention, to foci of apoptosis, which may occur, for example, within tumors either spontaneously, or in response to therapy.

In another embodiment of the invention, there is provided a compound which is selectively retained within a dying cell, represented by the structure set forth in formula (II):

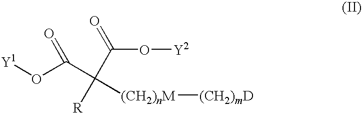

including pharmaceutically acceptable salts hydrates, solvates and metal chelates of the compound represented by the structure set forth in formula (II) and solvates and hydrates of the salts; wherein Y¹ is selected from hydrogen, C₁, C₂, C₃, C₄, C₅ and C₆ linear or branched alkyl, or an optionally substituted aryl or heteroaryl; Y² is selected from hydrogen, C₁, C₂, C₃, C₄, C₅ and C₆ linear or branched alkyl, or an optionally substituted aryl or heteroaryl; at least one of Y¹ or Y² is other than hydrogen; R represents hydrogen or C₁, C₂, C₃, C₄, C₅, C₆, C₇, C₈, C₉, C₁₀, C₁₁, C₁₂, C₁₃, C₁₄, C₁₅, C₁₆, linear or branched alkyl, linear or branched hydroxy-alkyl, linear or branched fluoro-alkyl, aryl or heteroaryl composed of one or two rings, or combinations thereof; n and m each stands for an integer of 0, 1, 2, 3 or 4; n and m may be the same or different; M is selected from null, hydrogen, —O—, —S—, and —N(U), wherein U stands for null, hydrogen, C₁, C₂, C₃, or C₄ alkyl; D is hydrogen or a marker for diagnostics. The marker for diagnostics may be in an embodiment of the invention a marker for imaging such as F, wherein the F may be ¹⁸F or ¹⁹F or a labeled metal chelate; the marker for imaging may be detected by color, fluorescence, x-ray, CT scan, magnetic resonance imaging (MRI) or radio-isotope scan such as single photon emission tomography (SPECT) or positron emission tomography (PET). Alternatively, D is a drug to be targeted to the dying cells, as define above.

In another embodiment of the invention there is provided a compound represented by the structure set forth in formula (III):

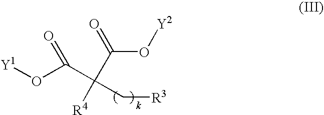

including pharmaceutically acceptable salts, hydrates, solvates and metal chelates of the compound represented by the structure set forth in formula (III) and solvates and hydrates of the salts; wherein R³ is ¹⁸F, ¹⁹F or hydroxyl; R⁴ is hydrogen, C₁, C₂, C₃, C₄, C₅, C₆, C₇, C₈, C₉ or C₁₀ linear or branched alkyl, and k is an integer selected from 0, 1, 2, 3, 4, 5, 6, 7, 8, 9, 10; Y¹ is selected from hydrogen, C₁, C₂, C₃, C₄, C₅ and C₆ linear or branched alkyl; Y² is selected from hydrogen, C₁, C₂, C₃, C₄, C₅ and C₆ linear or branched alkyl; at least one of Y¹ or Y² is other than hydrogen. In a preferred embodiment, Y¹ and Y² are each an ethyl group.

In another embodiment of the invention there is provided a compound represented by the structure set forth in formula (IV):

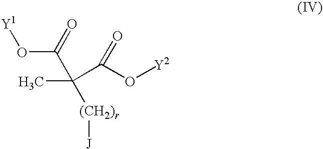

including pharmaceutically acceptable salts hydrates, solvates and metal chelates of the compound represented by the structure set forth in formula (IV) and solvates and hydrates of the salts; wherein J is —¹⁸F, —¹⁹F, —OH or a fluorescent group, and r stands for an integer of 1, 2, 3, 4, 5, 6, 7, 8, 9 and 10; Y¹ is selected from hydrogen, C₁, C₂, C₃, C₄, C₅ and C₆ linear or branched alkyl, or an optionally substituted aryl or heteroaryl; Y² is selected from hydrogen, C₁, C₂, C₃, C₄, C₅ and C₆ linear or branched alkyl, or an optionally substituted aryl or heteroaryl; at least one of Y¹ or Y² is other than hydrogen. In a preferred embodiment, Y¹ and Y² are each an ethyl group. In another embodiment, J is a dansyl group. In the case that J is a dansyl group, r=5, and Y¹ and Y² are each a methyl group, the compound is designated NST-ML-M.

In yet another embodiment of the invention, there is provided a compound represented by the structure set forth in formula (V):

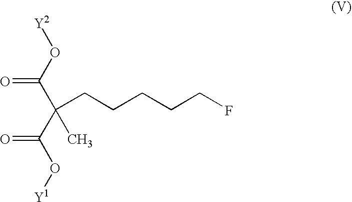

including pharmaceutically acceptable salts hydrates, solvates and metal chelates of the compound represented by the structure set forth in formula (V) and solvates and hydrates of the salts; wherein Y¹ is selected from hydrogen, C₁, C₂, C₃, C₄, C₅ and C₆ linear or branched alkyl, or an optionally substituted aryl or heteroaryl; Y² is selected from hydrogen, C₁, C₂, C₃, C₄, C₅ and C₆ linear or branched alkyl, or an optionally substituted aryl or heteroaryl; at least one of Y¹ or Y² is other than hydrogen; F is either ¹⁸F or ¹⁹F.

In yet another embodiment of the invention, there is provided a compound represented by the structure set forth in formula (VI):

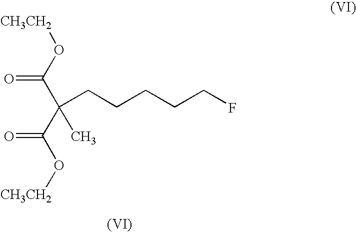

including pharmaceutically acceptable salts hydrates, solvates and metal chelates of the compound represented by the structure set forth in formula (VI) and solvates and hydrates of the salts; wherein F is either ¹⁸F, ¹⁹F.

In another embodiment of the invention, each of the compounds represented by formulae I, II, III, IV, V or VI may comprise or may be linked to a marker for diagnostics such as, for example, without being limited, Tc, Tc=O, In, Cu, Ga, Xe, Ti, Re and Re=O, ¹²³I, ¹³¹I, Gd(III), Fe(III), Fe₂O₃, Fe₃O₄, Mn(II) ¹⁸F, ¹⁵O, ¹⁸O, ¹¹C, ¹³C, ¹²⁴I, ¹³N, ⁷⁵Br, Tc-99m or In-111.

In another embodiment of the invention, there is provided a method of detecting dying cells within a cell population, the method comprising: (i). contacting the cell population with a compound represented by any one of the structure set forth in formulae I, II, III, IV, V or VI, or pharmaceutically acceptable salts, metal chelates, solvates and hydrates of the compound represented by the structure set forth in formulae I, II, III, IV, V or VI, and solvates and hydrates of the salts; and (ii). determining the amount of the compound bound to the cells, wherein a significant amount of compound retained within a cell after a certain period of time is indicative of the cell being a dying cell.

The term “significant amount of the compound bound to a cell” refers according to the invention to the amount of the compound of the invention, comprising or is being attached to a marker for diagnostics, which binds to a dying cell in an amount which is at least 30% greater than the amount bound to a normal cell after a certain time period, as was defined hereinabove. In another embodiment, the amount is higher by 50%. In another embodiment of the invention, the amount is higher by 75%. In another embodiment, the amount is higher by 150%. In another embodiment the amount is higher by about two-fold. In another embodiment the amount is higher than at least two-fold. In another embodiment, the amount is higher than at least five-fold. In another embodiment, the amount is higher by at least ten-fold.

In an embodiment of the invention, in reference to use of the compounds of the invention for obtaining images of cells undergoing a death process in a patient via radio-nuclide imaging by PET or SPECT, the calculation of the ratio between the amount of the compound bound to the dying cells versus. the amount bound to normal cells may be conducted by comparing the amplitude or intensity of the signal obtained from the tissue inflicted by the death process, with the amplitude/intensity obtained from an organ or tissue not inflicted by the death process.

According to another aspect of the invention, there is provided a method for detecting dying cells in a patient or an animal, the method comprising: (i) administering to the patient or animal a compound represented by the structure set forth in formulae I, II, III, IV, V or VI, wherein the compound comprises a marker for imaging, such as, for example, without limitation, ¹⁸F, or pharmaceutically acceptable salts, metal chelates, solvates and hydrates of the compound represented by the structure set forth in formulae I, II, III, IV, V or VI, and solvates and hydrates of the salts; and (ii) imaging the examined patient or animal, so as to determine the amount of compound bound to cells, wherein detection after a certain period of time of a significant amount of compound bound to cells indicates that these cells are dying cells.

In a non-limiting hypothesis, the mechanism of action of the compounds of the invention in retention within dying cells may potentially comprise, at least in part, the following steps:
(i). Passage through the cell membrane, due to the hydrophobic, uncharged structure of the compounds;
(ii). A fraction of molecules remains esterified, while another fraction undergoes partial or full cleavage of the ester moieties, with formation of the respective free mono- or di-acid forms, having one or two negative charges in physiological conditions. Upon capturing of a single proton in the di-acid form, a symmetrical hydrogen bond is formed, with the proton being “shared” between the two carboxylate groups of the malonate moiety, and with the remaining negative charge evenly distributed over the four carboxylate oxygen atoms. Charge dispersion therefore occurs and the ionic radius becomes significantly enlarged.
(iii). Clearance of the compound from viable cells while being retained within the dying cells. Movement is facilitated by the hydrophobic nature of the fully esterified molecules, or by the dispersion of the negative charge of the charged species. This movement is determined by the membrane potential map (MPM), and by the integrity of the plasma membrane. In viable cells, the trans-membrane potential, being positive outside the cell, causes an outward movement from the cell of either the polar species of the esterified compounds of the invention or the respective negatively-charged amphipathic free acids. In cells with disrupted cell membranes, the compounds will exit the cell through the membrane defects. By contrast, in cells undergoing apoptosis, the preservation of membrane continuity, along with the loss of trans-membrane potential will act to augment the retention of the compound within the dying cell.
   The compounds of the invention may be used for selective targeting of medicinally-useful agents to tissues and organs comprising dying cells, employing, without limiting, one of two approaches, of the invention:
(i). According to a first approach, termed hereinafter the “detection approach”, the selective retention or binding may be utilized for targeting a marker for imaging dying cells. This may be used in clinical practice, either in vivo, ex vivo or in vitro, for the diagnosis of diseases in which such cells emerge, as will be explained herein below.
(ii). According to a second approach, termed hereinafter the “therapeutic approach”, the property of selective retention or binding is used for selective targeting of therapeutic agents to organs and tissues in the body comprising dying cells, e.g., regions of cell death or neurodegeneration.

In accordance with the detection approach, the invention concerns a composition comprising a DCRC as an effective ingredient, comprising or linked to a marker for imaging, for the detection of dying cells either in vitro, ex vivo or in vivo. Such a DCRC is hereinafter designated “diagnostic DCRC”. The diagnostic DCRC is capable of performing selective retention within or binding to dying cells present in the assayed sample. Thereafter, the retention or binding may be identified by any means known in the art. The diagnostic DCRC of the invention enables targeting by the DCRC of the marker to the dying cells in a selective manner. Thereafter, the detectable label can be detected by any manner known in the art, and in accordance with the specific label used, for example, fluorescence, radioactive emission, color production, MRI, x-ray and the like. In one embodiment, the diagnostic DCRC is linked to the detectable label by a covalent or a non-covalent (e.g., electrostatic) binding.

In an embodiment of the invention, the detectable label may be any of the respective radio-isotopes of the metal ions Tc, oxo-Tc, In, Cu, Ga, Xe, Tl, Re, oxo-Re or the covalently linked atoms: ¹²³I and ¹³¹I for radio-isotope scans such as SPECT; Te, Gd(III), Fe(III) or Mn(II) for MRI; and ¹⁸F, ¹⁵O, ¹⁸O, ¹¹C, ¹³C, ¹²⁴I, ¹³N or ⁷⁵Br for positron emission tomography (PET) scan.

In an embodiment of the invention, the DCRC of the invention is aimed at clinical imaging of apoptosis via PET scan, and the DCRC comprises ¹⁸F atom(s).

Due to the short half-life of certain radio-isotopes used as markers for imaging, such as ¹⁸F, the attachment of such a marker for the purposes of clinical PET imaging may be performed immediately before the administration of the diagnostic compound to the patient. Therefore, it may be useful to synthesize a DCRC-PET precursor, comprising a moiety to be substituted by the radio-isotope such as ¹⁸F before administration to the patient. In one embodiment, the moiety to be replaced by ¹⁸F is selected from a hydroxyl group, a nitro group, a halogen atom, or an optionally substituted sulfonate group, such as mesylate, tosylate, or triflate. Such a DCRC-precursor or DCRC-PET precursor is also included in the scope of the invention.

The method for labeling a DCRC, which may be any DCRC of the structures described hereinabove, with ¹⁸F for PET imaging, comprises the step of attaching an ¹⁸F atom to the DCRC, thereby radio-labeling the DCRC with ¹⁸F for PET imaging. Optionally, the functional groups of the DCRC may be protected by appropriate protecting groups prior to the step of attaching the ¹⁸F atom. The protecting groups are thereafter optionally removed after the step of attachment of the ¹⁸F atom. Such DCRC, having protected functional groups, are also included in the scope of the invention.

In the case that the marker is a metal atom (e.g., Gd, ^{99m}Tc or oxo-^{99m}Tc for MRI or SPECT, respectively), the DCRC may comprise a metal chelator. The metal coordinating atoms of the chelator may be nitrogen, sulfur or oxygen atoms. In an embodiment of the invention, the chelator may be, for example, diaminedithiol, monoamine-monoamide-bisthiol (MAMA), triamide-monothiol, or monoamine-diamide-monothiol. In such a case, both a DCRC-chelate precursor, being the DCRC attached to or comprising a chelator prior to complexation with the metal atom, and the complex comprising the metal atom, are included in the scope of the invention.

For fluorescent detection, the diagnostic DCRC may comprise a fluorescent group selected from any fluorescent probe known in the art. Examples of such probes are 5-(dimethylamino) naphthalene-1-sulfonylamide (dansyl-amide), and fluorescein.

The compounds of the invention may be used for the detection and diagnosis of a wide variety of medical conditions, characterized by dying cells.

Examples of clinical conditions characterized by dying cells are as follows: Diseases which are characterized by occurrence of excessive apoptosis, such as degenerative disorders, neurodegenerative disorders (e.g., Parkinson's disease, Alzheimer's disease, Huntington chorea), AIDS, Motor Neuron Diseases such as amyotrophic lateral sclerosis (ALS), Prion Diseases (e.g. Creutzfeldt-Jacob disease), myelodysplastic syndromes, ischemic or toxic insults, graft cell loss during transplant rejection; tumors, and especially highly malignant/aggressive tumors, are also often characterized by increased apoptosis in addition to excessive tissue proliferation.

Vascular disorders, such as myocardial infarction, cerebral stroke, deep vein thrombosis, disseminated intravascular coagulation (DIC), thrombotic thrombocytopenic purpura (TTP), sickle cell diseases, thalassemia, antiphospholipid antibody syndrome, systemic lupus erythematosus.

Inflammatory disorders, and/or diseases associated with immune-mediated etiology or pathogenesis; auto-immune disorders such as antiphospholipid antibody syndrome, systemic lupus erythematosus, connective tissue disorders, such as rheumatoid arthritis, scleroderma; thyroiditis; dermatological disorders such as pemphigus or erythema nodosum; autoimmune hematological disorders; autoimmune neurological disorders such as myasthenia gravis, multiple sclerosis, inflammatory bowel disorders, such as ulcerative colitis and vasculitis.

Atherosclerotic plaques, and especially plaques that are unstable, vulnerable and prone to rupture, are also characterized by dying cells, such as apoptotic macrophages, apoptotic smooth muscle cells or apoptotic endothelial cells.

The methods of the present invention may be used to detect and/or diagnose one or more of the aforementioned clinical conditions in a human patient or animal subject. Additionally, the detection may also be carried out in a person already known to have the respective disease, for the purpose of evaluating the disease severity or in order to monitor the disease course and/or response to various therapeutic modalities. A non-limited example for such monitoring is evaluation of response to anticancer therapy. Since most anti-tumor treatments, such as chemotherapy or radiotherapy, exert their effect by induction of apoptosis, detection by a diagnostic DCRC of therapy-induced apoptosis of tumor cells may teach about the extent of sensitivity of the tumor to the anti-tumor agent. This may substantially shorten the lag period between the time of administration of the anti-cancer treatment and the time of proper assessment of its efficacy.

Moreover, the detection may also be used to monitor adverse effects of anti-cancer treatments. A large part of such adverse effects is due to untoward treatment-induced apoptosis in normal, yet sensitive cells, such as those of the gastrointestinal epithelium or the bone marrow hematopoietic system.

In addition, the detection may aim at characterization of intrinsic apoptotic load within a tumor, often correlated with the level of tumor aggressiveness; and may also assist in the detection of metastases, via detection of the intrinsic apoptosis frequently occurring within metastases.

Similarly, the diagnostic DCRCs of the present invention may be useful in monitoring graft survival after organ transplantation, since apoptosis plays a major role in cell loss during graft rejection.

In addition, the detection may aim at monitoring the response to cyto-protective treatments, and thus aid in screening and development of drugs, which are capable of inhibiting cell loss in various diseases (for example those recited above) by enabling a measure of assessment of cell death.

The detection may also be useful for the detection of atherosclerotic plaques, since destabilization of such plaques, rendering them vulnerable, prone to rupture, thrombosis and embolization, is characterized by participation of several types of dying cells, including apoptotic cells (apoptotic macrophages, smooth muscle cells and endothelial cells).

In accordance with this approach, the invention discloses a method of detection of dying cells in a cell population, derived from a whole body, an organ, a tissue, or a tissue culture, the method comprising: (i). contacting the cell population with a diagnostic DCRC according to any of the embodiments of the invention; and (ii). determining after a certain period of time the amount of DCRC bound to the cell population, wherein detection of a significant amount of compound after a certain period of time, bound to a cell within the cell population indicates that the cell is a dying cell.

In another embodiment, the invention further relates to a method for detecting dying cells in a patient or in an animal in vivo, the method comprising: (i). administering a diagnostic DCRC to the examined patient or animal; the administration being performed by any means known in the art, such as parenteral (e.g., intravenous) or oral administration; and (ii). imaging the examined patient or animal, after a certain period of time by any method known in of the art (e.g., PET scan, SPECT, MRI), to detect and determine the amount of diagnostic DCRC bound to cells, wherein a significant amount of compound bound to a cell indicates that the cell is a dying cell.

In another embodiment, the invention is related to a method for the detection of dying cells in a tissue or cell culture sample in vitro or ex-vivo, the method comprising: (i). contacting the sample with a diagnostic DCRC, which may be any of the DCRC compounds described in the invention, under conditions enabling binding of the diagnostic DCRC to the biological membranes of dying cells; and (ii). detecting after a certain period of time the amount of diagnostic DCRC bound to the cells; the presence of a significant amount of bound diagnostic compound indicating the presence of dying cells within the tissue or cell culture.

The step of detection in the in vitro or ex-vivo studies may be, for example, in the case of a fluorescent-labeled compound of the invention, without limitation, by using flow cytometric analysis, which permits cell visualization utilizing equipment that is widely commercially available.

The term “significant amount” according to the invention, means that the amount of DCRC retained in or bound to a dying cell is at least 30% higher than the amount bound to a non-dying cell after a certain period of time. The actual amount may vary according to the imaging method and equipment utilized, and according to the organs or tissues examined. In another embodiment, the amount of DCRC retained or bound to a dying cell is at least 50% higher than the amount bound to a non-dying cell. In another embodiment the amount of DCRC bound to a dying cell is at least 75% higher than the amount bound to a non-dying cell. In another embodiment the amount of DCRC bound to a dying cell is at least twice the amount bound to a non-dying cell. In another embodiment, the amount of DCRC bound to the dying cell is at least four times the amount bound to a non-dying cell. In another embodiment the amount of DCRC bound to a dying cell is at least six times the amount bound to a non-dying cell. In another embodiment the amount of DCRC bound to a dying cell is at least eight times the amount bound to a non-dying cell. In another embodiment the amount of DCRC bound to a dying cell is at least ten times the amount bound to a non-dying cell.

The method of the invention may also be used for monitoring the effects of various therapeutic modalities used for treatment of diseases or medical conditions, or alternatively for basic science research purposes as explained above.

In accordance with a second approach of the invention, termed “the therapeutic approach”, the invention concerns a pharmaceutical composition comprising a DCRC, used for targeting an active drug or a pro-drug to dying cells.

A “therapeutic DCRC” according to the invention means a DCRC comprising a drug or a DCRC being conjugated to a medicinally-useful agent. The term “conjugate” means two molecules being linked together by any means known in the art.

The association between the medicinally-useful drug and the DCRC, wherein the drug is comprised or linked to the therapeutic DCRC, may be by covalent binding, by non-covalent binding (e.g., electrostatic forces) or by formation of carrier particles (such as liposomes), comprising the drug and having on their surface a DCRC which targets the conjugate to the dying cells. Once the drug reaches the target, it should be able to exert its physiological activity, either when still being part of the DCRC-conjugate, after disconnecting from the DCRC unit (for example by cleavage, destruction, etc., activity of natural enzymes), by phagocytosis of drug-containing liposomes having DCRC on their membrane, or by any other known mechanism.

The drug should be chosen in accordance with the specific disease for which the composition is intended.

Pharmaceutical compositions for therapeutics, as well as diagnostic compositions of the invention may be administered by any of the known routes, inter alia, oral, intravenous, intraperitoneal, intramuscular, subcutaneous, sublingual, intraocular, intranasal or topical administration, or intracerebral administration. The carrier should be selected in accordance with the desired mode of administration, and include any known components, e.g. solvents; emulgators, excipients, talc; flavors; colors, etc. The pharmaceutical composition may comprise, if desired, other pharmaceutically-active compounds which are used to treat the disease, eliminate side effects or augment the activity of the active component.

In accordance with the therapeutic approach, the invention still further concerns a method for treating a disease manifesting dying cells, comprising administering to an individual in need of such treatment an effective amount of a therapeutic DCRC, the therapeutic DCRC comprising a drug being active as a treatment for the disease or a pro-drug to be converted to an active drug in vivo, typically in the targeted area. The therapeutic DCRC allows for selective targeting of the drug to the tissues comprising dying cells, thus augmenting its local concentration, and potentially enhancing its therapeutic effect at the target site. Such medical disorders may be disorders as defined above.

In another embodiment, there is provided a method of killing cancer cells in a tumor, comprising the step of targeting apoptotic cells within the tumor by administration of a therapeutic DCRC, comprising any one of the compounds set forth in formulae I, II, III, IV, V or VI and a cytotoxic drug, thereby killing the cancer cells.

The term “effective amount” of the therapeutic DCRC refers to an amount capable of decreasing, to a measurable level, at least one adverse manifestation of the disease, and should be chosen in accordance with the drug used, the mode of administration, the age and weight of the patient, the severity of the disease, etc.

In another embodiment, the therapeutic DCRC of the invention comprises or is being linked to a radioisotope which has a therapeutic effect. An example, without limitation, for such a radio-isotope is Yttrium 90, Iodine 131, Rhenium 188, Holmium 166, Indium 111, Leutitium 177, or any other radiation-emitting radioisotopes, useful for therapeutic purposes.

### EXAMPLES

In order to understand the invention and to see how it may be carried-out in practice, the following examples are described: an example directed to synthesis of the compounds of the invention; and an example directed to the performance of the compounds of the invention in selective binding to cells undergoing apoptosis. In order to allow detection of the compounds of the invention, they were labeled by attachment to a fluorescent group, i.e., dansylamide, and detected by fluorescent microscopy. Presented are results with NST-ML-M, according to formula (IV). Detection was performed by fluorescent microscopy, utilizing the inherent fluorescent properties of the dansyl group. The selectivity of binding of the compounds to the apoptotic cells was examined in vivo, in a murine model of carcinoma.

### Example 1

### Synthesis of 2-methyl-2(3-fluorobutyl)-malonate (NST-ML-10E, Scheme 1)

4-bromo-1-butanol (1), 3 g, was treated with 1.5 eq of 3,4-dihydro-2H-pyran and 0.1 eq of pyridinium para tuloenesulfonate (PPTS) in 135 mL of CH₂Cl₂. After work-up and purification, 1.45 g (33%) of product 2 was obtained. 1.0 eq of diethylmethylmalonate was deprotonated with 1 eq of NaH and 1.0 eq of bromide 2 was added along with catalytic amount of KI at 50° C. A complete conversion was observed after 10 hours and a 90% yield was obtained. Deprotection of tetrahydro pyran (THP) with PPTS in ethanol at 55° C. went smoothly. After work-up, a quantitative yield of alcohol 4 was obtained and directly used for the mesylation reaction. With the mesylate 5 in hand, a kryptofix-mediated fluorination was performed. Compound 6 was obtained in 68% yield.

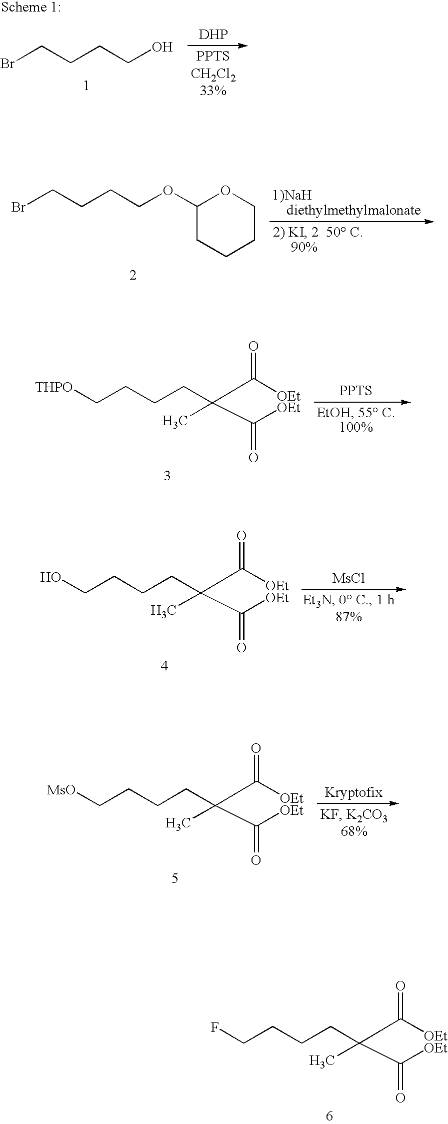

### Example 2

### Selective Accumulation of NST-ML-M in Cells Undergoing Apoptosis Induced by Treatment with an Anti-Cancer Agent

Many chemotherapeutic agents exert their anti-cancer effect via induction of apoptosis in the tumor cells. In addition to this desired apoptosis-inducing effect on the tumor cells, an adverse effect of the administration of these chemotherapeutic agents is associated with induction of apoptosis in normal tissues, such as the epithelium of the small intestine. We therefore examined whether NST-ML-M, administered intravenously, can detect this chemotherapy-induced apoptosis in a tumor and small intestine tissues.

Female athymic 8-10 weeks old nude mice (Harlan laboratories, Jerusalem) were used. Human colon adenocarcinoma (Colo-205) cells were inoculated subcutaneously. Solitary tumors were generated by following the injection of 10⁶ viable cells in 100 μl HBS into the flank. Upon tumor growth to 8 mm in diameter, mice were treated intraperitonealy with cyclophosphamide and taxol. For the treatment, cyclophosphamide (Sigma, Israel) was dissolved in saline, and injected at a dose of 300 mg/Kg of body weight. Taxol (Mead Johnson, USA) was diluted in saline and was injected at a dose of 20 mg/Kg of body weight.

One day after administration of chemotherapy, mice were injected intravenously with NST-ML-M, 70 mg/Kg or 140 mg/Kg of body weight (the agent was dissolved in 10% cremophore & NaPPi buffer 0.1M, pH 7.4). Sixty minutes later, mice were sacrificed, and tumors and sections of the small intestine were excised, frozen in liquid nitrogen, and then subjected to preparation of frozen sections and fluorescent microscopy, to detect the retention of NST-ML-M in its target sites, through the detection of the inherent fluorescent properties of the dansyl group.

### Experimental Results

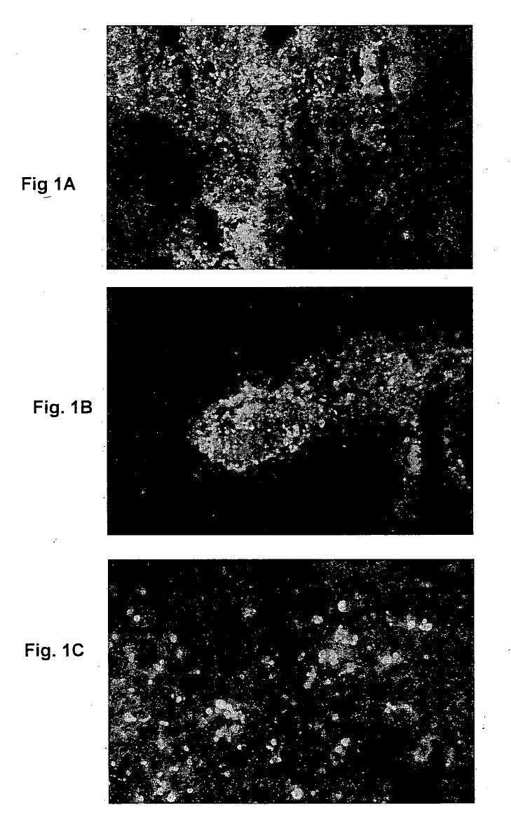

As shown in FIGS. 1A and 1B, following intravenous administration to the chemotherapy-treated mice, NST-ML-M was selectively retained within the tumors, in specific cells that were further identified as cells undergoing cell death by the TUNEL staining for apoptotic DNA fragmentation. In contrast, the compound was not retained in viable cells. This phenomenon was evident in both 70 mg/Kg and 140 mg/Kg doses. In addition to retention in the tumor, selective retention of the compound was also observed in cells undergoing apoptosis in the crypta of the small intestine epithelium in response to the systemic administration of chemotherapy. The identity of these cells as apoptotic cells was also confirmed by the TUNEL staining. By contrast, the compound was not retained in the adjacent viable cells (FIG. 1C). These results, from both the tumor and the intestine in animals treated with chemotherapy, show the capability of NST-ML-M to serve as a probe for selective detection of apoptosis in vivo.

It is to be understood that the invention is not limited in its application to the details set forth in the description contained herein or illustrated in the drawings. The invention is capable of other embodiments and/or being practiced and carried out in various ways. Those skilled in the art will readily appreciate that various modifications and changes can be applied to the embodiments of the invention as hereinbefore described, without departing from its scope, as defined in and by the appended claims.

1.-35. (canceled)

36. A method for targeting a compound to cells undergoing a cell death process in a cell population comprising the steps of:
(i) contacting the cell population with a compound represented by the structure set forth in formula (I): 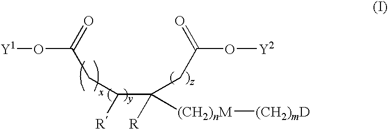
wherein Y¹ and Y² are each independently selected from hydrogen, C₁, C₂, C₃, C₄, C₅ and C₆ linear or branched alkyl, aryl or heteroaryl, at least one of Y¹ or Y² is other than hydrogen;
each of the R and R′ groups is independently selected from, hydrogen, C₁, C₂, C₃, C₄, C₅, C₆, C₇, C₈, C₉, C₁₀, C₁₁, C₁₂, C₁₃, C₁₄, C₁₅, C₁₆, linear or branched alkyl, aryl or heteroaryl composed of one or two rings;
n and m each stands independently for an integer of 0, 1, 2, 3 or 4;
M is selected from null, —O—, or —S—;
x and z each stands independently and is an integer of 0, 1 or 2;
y is an integer of 0, 1 or 2, wherein when y=0, R′ is null; and
D is selected from a group consisting of hydrogen, hydroxyl, a marker for diagnostics or a drug.
(ii) thereby targeting said compound to the cells undergoing a cell death process within the cell population.

37. The method according to claim 36 wherein the marker for diagnostics is selected from ¹⁸F or a radio-labeled metal chelate.

38. The method according to claim 36, comprising contacting the cell population with a compound represented by the structure set forth in formula (I), wherein M is null.

39. A method for targeting a compound to cells undergoing a cell death process in a cell population, comprising the steps of:
i. contacting the cell population with a compound or a conjugate comprising said compound, wherein said compound is represented by the structure set forth in formula (II): 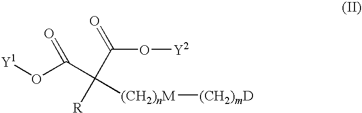
wherein Y¹ and Y² are each independently selected from hydrogen, C₁, C₂, C₃, C₄, C₅ and C₆ linear or branched alkyl, aryl or heteroaryl, at least one of Y¹ or Y² is other than hydrogen;
R represents hydrogen or C₁, C₂, C₃, C₄, C₅, C₆, C₇, C₈, C₉, C₁₀, C₁₁, C₁₂, C₁₃, C₁₄, C₁₅, C₁₆, linear or branched alkyl, aryl or heteroaryl composed of one or two rings;
n and m each stands independently for an integer of 0, 1, 2, 3 or 4;
M is selected from null, —O—, —S—, and —N(U)-, wherein U stands for hydrogen, C₁, C₂, C₃, or C₄ alkyl and
D is selected from a group consisting of hydrogen, hydroxyl, a marker for diagnostics or a drug.

40. The method according to claim 39, comprising contacting the cell population with a compound represented by the structure set forth in formula (II), wherein M is null.

41. A compound represented by the structure set forth in formula (II): 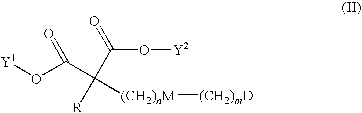
wherein Y¹ and Y² are each independently selected from hydrogen, C₁, C₂, C₃, C₄, C₅ and C₆ linear or branched alkyl, aryl or heteroaryl, at least one of Y¹ or Y² is other than hydrogen;
R represents hydrogen or C₁, C₂, C₃, C₄, C₅, C₆, C₇, C₈, C₉, C₁₀, C₁₁, C₁₂, C₁₃, C₁₄, C₁₅, C₁₆, linear or branched alkyl, aryl or heteroaryl composed of one or two rings;
n and m each stands for an integer of 0, 1, 2, 3 or 4;
M is selected from null, —O—, —S—, and —N(U)-, wherein U stands for hydrogen, C₁, C₂, C₃, or C₄ alkyl; and
D is a marker for diagnostics or a drug.

42. The compound according to claim 41 wherein said marker for diagnostics is detectable by fluorescence, x-ray, CT scan, magnetic resonance imaging (MRI), radio-isotope scan, single photon emission tomography (SPECT) or positron emission tomography (PET).

43. The compound according to claim 41 wherein said marker for diagnostics is selected from Tc, Tc=O, In, Cu, Ga, Xe, Tl, Re and Re=O, ¹²³I, ¹³¹I, Gd(III), Fe(III), Fe₂O₃, Fe₃O₄, Mn(II) ¹⁸F, ¹⁵O, ¹⁸O, ¹¹C, ¹³C, ¹²⁴I, ¹³N, ⁷⁵Br, Tc-99m or In-111.

44. The compound according to claim 41 represented by the structure set forth in formula IV 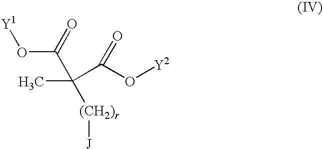
wherein Y¹ and Y² are each independently selected from hydrogen, C₁, C₂, C₃, C₄, C₅ and C₆ linear or branched alkyl, aryl or heteroaryl, at least one of Y¹ or Y² is other than hydrogen;
r stands for an integer of 0, 1, 2, 3, 4, 5, 6, 7 or 8; and
J is selected from the group consisting of ¹⁸F, hydroxyl, a fluorescent group or a drug.

45. The compound according to claim 44, wherein Y¹ and Y² are each independently selected from a methyl or ethyl group.

46. The compound according to claim 44, wherein J is an ¹⁸F radio-isotope or a dansyl group.

47. The compound according to claim 44 wherein r=5; and J is ¹⁸F.

48. The compound according to claim 44 wherein Y¹ and Y² are each an ethyl group; r=5; and J is ¹⁸F.

49. A compound represented by the structure set forth in formula (III): 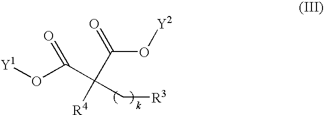
wherein R³ is a moiety to be substituted by an ¹⁸F radio-isotope upon radio-labeling, so as to generate an ¹⁸F-labeled PET compound;
R⁴ is selected from hydrogen, C₁, C₂, C₃, C₄, C₅, C₆, C₇, C₈, C₉ or C₁₀ linear or branched alkyl; and
k is an integer selected from 0, 1, 2, 3, 4, 5, 6, 7 or 8;
Y¹ and Y² are each independently selected from hydrogen, C₁, C₂, C₃, C₄, C₅ and C₆ linear or branched alkyl, aryl or heteroaryl, at least one of Y¹ or Y² is other than hydrogen.

50. The compound according to claim 49 wherein said moiety is a sulfonate, a nitro, a halogen, or a hydroxyl group.

51. The compound according to claim 50, wherein said sulfonate is selected from mesylate, tosylate and triflate.

52. The compound according to claim 49, wherein Y¹ and Y² are each independently selected from a methyl or ethyl group.
